(19) United States
(12) Patent Application Publication
(10) Pub. No.: US 2014/0039346 A1
(43) Pub. Date: Feb. 6, 2014

(54) DEVICE AND METHOD FOR COLLECTING TISSUE SAMPLES

(71) Applicant: Alexandra J. Gillespie, Dallas, TX (US)

(72) Inventor: Alexandra J. Gillespie, Dallas, TX (US)

(21) Appl. No.: 13/956,876

(22) Filed: Aug. 1, 2013

Related U.S. Application Data
(60) Provisional application No. 61/679,962, filed on Aug. 6, 2012.

(51) Int. Cl. A61B 10/02

(52) U.S. Cl. CPC A61B 10/0291
USPC 600/569

(57) ABSTRACT

A tissue sampling device includes a single wire spirally wrapped at its half-length. This wire is partially covered in a hollow flexible tube. Located along the distal end portion of the wire is a brush with bristles that are used for collecting a tissue sample. An atraumatic bulb is located on the extreme distal end of the wire to protect the tissue during insertion. The flexible tube is contained within a sheath to cover the brush during insertion and after tissue collection. The sheath is moved back to expose the brush and allow for collection of a tissue sample. The flexible tube is scored in centimeter gradations to allow measurement of the insertion depth of the device. The atraumatic bulb may be more elongated than round, such as egg-shaped, elliptical, oblong, ovate, ovoid, pear-shaped, bullet-shaped, and the like.

### CROSS-REFERENCE TO RELATED APPLICATIONS

This application claims the benefit of priority to, and hereby incorporates by reference, U.S. Provisional Application Ser. No. 61/679,962, entitled “Device and Method for Collecting Tissue Samples,” filed Aug. 6, 2012.

### BACKGROUND OF THE INVENTION

1. Technical Field of Invention

The present invention relates generally to medical sampling devices and in particular to a device and method for collecting tissue samples

2. Description of Related Art

Within the medical industry there are a variety of devices for sampling tissue from various sites of the human body. Sampling of the endometrium has a long history of utilizing different samplers ranging from curettes and pipelles to various brushes and simple swabs. Historically, all of these devices have their limitations. For example, the curette can obtain a significant amount of tissue from the endometrium. However, it requires a surgical setting, anesthesia, and can cause possible permanent scarring of the endometrial lining which can lead to infertility. The Pipelle technique involves aspiration of the tissues sample and can be done on an outpatient basis. However, diffuse sampling of the endometrium is limited. Different brushes are used and have limitations such as large size which makes tissue collection uncomfortable for the patient or brushes having the inability to exfoliate sufficient cells for examination or inability to capture the exfoliated cells, or there are not indicators on the depth of placement of the brush into the cavity, and/or brushes can cause contamination of the sample by other body sites such as the cervix and the vagina. Furthermore, there is no provision among known existing brushes for adjusting the brush to accommodate differing anatomy.

Because current technology provides a single brushhead length and limited bristle length, there is a compromise between patient comfort and collection effectiveness. Therefore, a need exists for an improved device and method for collecting tissue samples.

All references cited herein are incorporated by reference to the maximum extent allowable by law. To the extent a reference may not be fully incorporated herein, it is incorporated by reference for background purposes and indicative of the knowledge of one of ordinary skill in the art.

### SUMMARY OF THE INVENTION

The problems presented in existing tissue sampling devices are solved by the device and method of the present invention. In accordance with one embodiment a tissue sampling device is provided. The tissue sampling device includes a spirally twisted flexible wire with opposed proximal and distal ends. Also included is a plastic tube covering a significant portion of the wire to provide additional rigidity without making the overall brush stiff. Along the distal end portion of the wire is a brush that includes bristles that were used for collecting a tissue sample. The bristles are fixed within the spirally twisted wire near the distal end and are tapered from smaller to larger towards the distal end of the wire. Tapering of the bristles from the distal end of the device allows for more global tissue collection of the endometrium because of the shape of the endometrial cavity. An atraumatic bulb is located on the extreme distal end of the twisted wire. The atraumatic bulb aids in insertion into the cervical area without trauma and helps prevent further trauma to the uterus. In some embodiments, the atraumatic bulb may have a round or spherical shape for ease of insertion of the tissue sampling device into the cervical area. In some embodiments, to further facilitate insertion of the tissue sampling device into the cervical area, the shape of the atraumatic bulb may be more elongated than round, such as egg-shaped, elliptical, oblong, ovate, ovoid, pear-shaped, bullet-shaped, and the like. The tissue sampling device also includes a handle on the extreme proximal end of the plastic core.

In accordance with another embodiment, the plastic tube and twisted wire are contained within a sheath of shorter length than the twisted wire, such that the sheath can be moved along the plastic tube to the atraumatic bulb on the distal end of the twisted wire, thereby covering the brush during insertion and removal after tissue collection. Before insertion, the sheath can be moved into position over the distal end of the twisted wire to protect the brush during insertion. Having the brush covered during insertion also increases comfort for the patient and protects the brush from collecting tissue from unintended areas. The sheath is moved back toward the proximal end of the twisted wire after the device has been inserted to the proper collection depth, exposing the brush and allowing for collection of a tissue sample. The sheath may be moved to completely uncover the brush or may be moved in gradients to uncover portions of the brush. This allows the practitioner to adjust the effective collection area of the brush based on the anatomy of the patient. The plastic tube covering the wire is scored in centimeter gradations along the plastic tube with markings indicating the exact length of the brush inserted into the uterus, starting from the distal tip of the brush to the proximal end of the plastic tube. This allows the clinician to know how deeply the brush is inserted into the uterus. The sheath is approximately the same length as the plastic tube and in position to cover the brush bristles prior to insertion. The sheath may be formed of a clear material such that the gradations on the plastic tube may be viewed through the sheath. The ability to measure insertion depth increases the certainty that the tissue sample collected is from the correct area, After a tissue sample is collected from the proper area, while the tissue sampling device remains inserted, the sheath can be moved back along the distal end of the twisted wire to cover the brush bristles before removing the brush. This allows for the tissue sample to be protected on the brush within the sheath during removal.

Additionally, the gradations along the flexible tube allow the practitioner to measure the length of bristles exposed. As the practitioner pulls the sheath from its insertion position towards the handle, the further the sheath is pulled the more bristles are exposed. The gradations (ruler) provide a visual confirmation of this measurement and allow the practitioner to be precise in exposing only a certain length of the brush bristles. This measurement allows the practitioner to have better control of where the tissue is sampled and allows the practitioner to adjust the length of brush based on patient specific parameters; such as uterine size measured during previous tests or inferred based on patient history. Control of brush exposure increases sampling precision and patient comfort.

Other objects, features, and advantages of the present invention will become apparent with reference to the drawings and detailed description that follow.

### DESCRIPTION OF THE DRAWINGS

The foregoing and other aspects of the disclosed embodiments will become apparent from the following detailed description and upon reference to the drawings, wherein:

FIG. 1A is a perspective view of a tissue sampling device in accordance with an embodiment of the present invention;

FIG. 1B is a close up of the distal end of the device in FIG. 1A with conically tapered brush bristles;

FIG. 1C is a close up of the distal end of the device in FIG. 1A with step tapered brush bristles;

FIG. 2A is a perspective view of a tissue sampling device in accordance with an embodiment with a sheath positioned to cover the brush bristles;

FIG. 2B is a close up of the distal end of the device in FIG. 2A with conically tapered brush bristles enclosed within the sheath;

FIG. 2C is a close up of the distal end of the device in FIG. 2A with step tapered brush bristles enclosed within the sheath;

FIG. 3A is a perspective view of a tissue sampling device in accordance with an embodiment with the sheath partially pulled back from the brush bristles;

FIG. 3B is a close up of the distal end of the device in FIG. 3A with conically tapered brush bristles;

FIG. 3C is a close up of the distal end of the device in FIG. 3A with step tapered brush bristles;

FIG. 4A is a perspective view of a tissue sampling device in accordance with an embodiment with the sheath pulled all the way back to fully expose the brush bristles;

FIG. 4B is a close up of the distal end of the device in FIG. 4A with conically tapered brush bristles;

FIG. 4C is a close up of the distal end of the device in FIG. 4A with step tapered brush bristles;

FIG. 5 is a view of the tissue sampling device inserted within a uterus with the brush totally covered by the sheath;

FIG. 6 is a view of the tissue sampling device inserted within a uterus with the brush partially exposed;

FIG. 7 is a view of the tissue sampling device inserted within a uterus with the brush fully exposed; and

FIGS. 8A-8C are top, cross-sectional, and perspective views, respectively, of an exemplary atraumatic bulb that may be used with the tissue sampling device.

### DESCRIPTION OF DISCLOSED EMBODIMENTS

The drawings described above and the written description of specific structures and functions below are presented for illustrative purposes and not to limit the scope of what has been invented or the scope of the appended claims. Nor are the drawings drawn to any particular scale or fabrication standards, or intended to serve as blueprints, manufacturing parts list, or the like. Rather, the drawings and written description are provided to teach any person skilled in the art to make and use the inventions for which patent protection is sought. Those skilled in the art will appreciate that not all features of a commercial embodiment of the inventions are described or shown for the sake of clarity and understanding.

Persons of skill in this art will also appreciate that the development of an actual, real-world commercial embodiment incorporating aspects of the inventions will require numerous implementation-specific decisions to achieve the developer's ultimate goal for the commercial embodiment. Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would nevertheless be a routine undertaking for those of skill in this art having the benefit of this disclosure.

It should also be understood that the embodiments disclosed and taught herein are susceptible to numerous and various modifications and alternative forms. Thus, the use of a singular term, such as, but not limited to, “a” and the like, is not intended as limiting of the number of items. Similarly, any relational terms, such as, but not limited to, “top,” “bottom,” “left,” “right,” “upper,” “lower,” “down,” “up,” “side,” and the like, used in the written description are for clarity in specific reference to the drawings and are not intended to limit the scope of the invention or the appended claims.

All references cited herein are incorporated by reference to the maximum extent allowable by law. To the extent a reference may not be fully incorporated herein, it is incorporated by reference for background purposes and indicative of the knowledge of one of ordinary skill in the art.

In the following detailed description of the preferred embodiments, reference is made to the accompanying drawings, which form a part hereof, and in which is shown by way of illustration specific preferred embodiments in which the invention may be practiced. These embodiments are described in sufficient detail to enable those skilled in the art to practice the invention, and it is understood that other embodiments may be utilized and that logical mechanical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

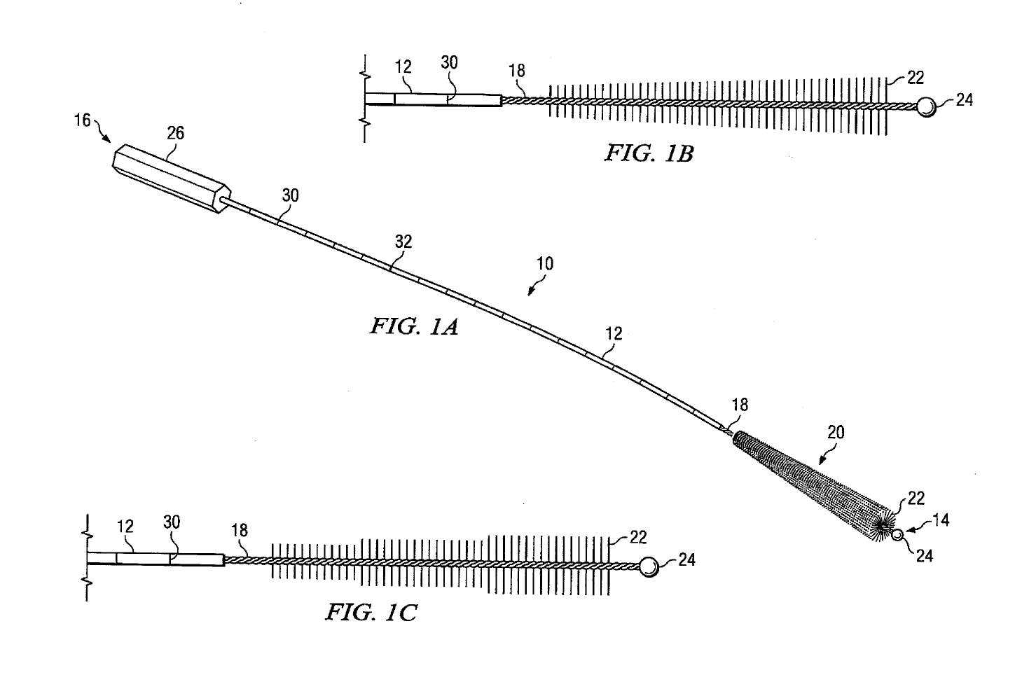

FIG. 1A is a perspective view of a tissue sampling device 10 in accordance with an embodiment of the present invention, FIG. 1B is a close up of the distal of the device 10 in FIG. 1A with conically tapered brush bristles 22, and FIG. 1C is a close up of the distal of the device 10 in FIG. 1A with step tapered brush bristles 22. A similar format is used in subsequent figures for consistency.

The distal end 14 of the tissue sampling device 10 shows twisted wire 18 extending from the plastic tube 12. In accordance with one embodiment the tissue sampling device 10 is used for uterine endometrial tissue collection. The tissue sampling device of the present embodiment includes an elongated twisted wire 18 having an opposed proximal end 16 and distal end 14. The length of the wire 18 in the present embodiment is approximately 28.5 cm, which has been found to be a functional length for the present application. However, one skilled in the art will understand that a wire 18 of different length may be used for different applications. The wire 18 should not be too rigid as to cause discomfort for the patient, but also cannot be so flimsy that proper insertion is made difficult. One skilled in the art will understand that the wire 18 can be made from a variety of flexible materials, such as stainless steel, and the like.

The twisted wire 18 is comprised of a single elongated wire that is contained within the tube 12. The wire 18 is spirally wrapped at its half-length such that the wire is folded over and the two half lengths are twisted together to form its final length. The wire 18 should be made of stainless steel to protect the health of the patient. While one skilled in the art will understand that the diameter of the wire can vary and still obtain its usefulness, the diameter must be such that the wire 18 can be spirally wrapped at its half-length and enclosed within a tube 12 having an approximate diameter of 3 mm. The distal end 14 of the wire 18 includes a brush 20 approximately 5.5 cm in length along the distal end 14 of the wire 18. The brush 20 includes bristles 22 fixed within the spirally wrapped wire 18 that protrude beyond the wire 18. The bristles 22 can be made from most materials that are used for current brush tissue sampling systems, such as nylon and other FDA approved materials for such devices.

The tissue sampling device 10 includes an atraumatic bulb 24 located on the distal end 14 of the wire 18. The bulb can be made from any biocompatible material, such as a flexible plastic, that will protect the surrounding tissue during collection. The device 10 also includes a handle 26 on the proximal end 16 of the wire 18. Any of a variety of handles can be used with the device. For example, a ring can be used which may help with removing the device after collection. As shown, handle 26 is a hexagonal cylinder, which may assist with rotating the device during tissue collection. In production, the twisted wire 18 and tube 12 are firmly attached to the handle 26 and may extend into the handle 26 to facilitate a firm connection.

Additionally, the plastic tube 12 may be scored or printed with centimeter gradations 30, such as every centimeter from the distal end 14 of the plastic tube 12 to the proximal end 16 of the plastic tube 12. In an embodiment, the gradations are numbered to show the distance from the distal tip of the device 10. An additional sheath mark 32 may be added among the gradations as is discussed in more detail below. The gradations 30 allows for insertion depth measurement and brush exposure measurement to ensure accurate tissue sampling in a variety of anatomies.

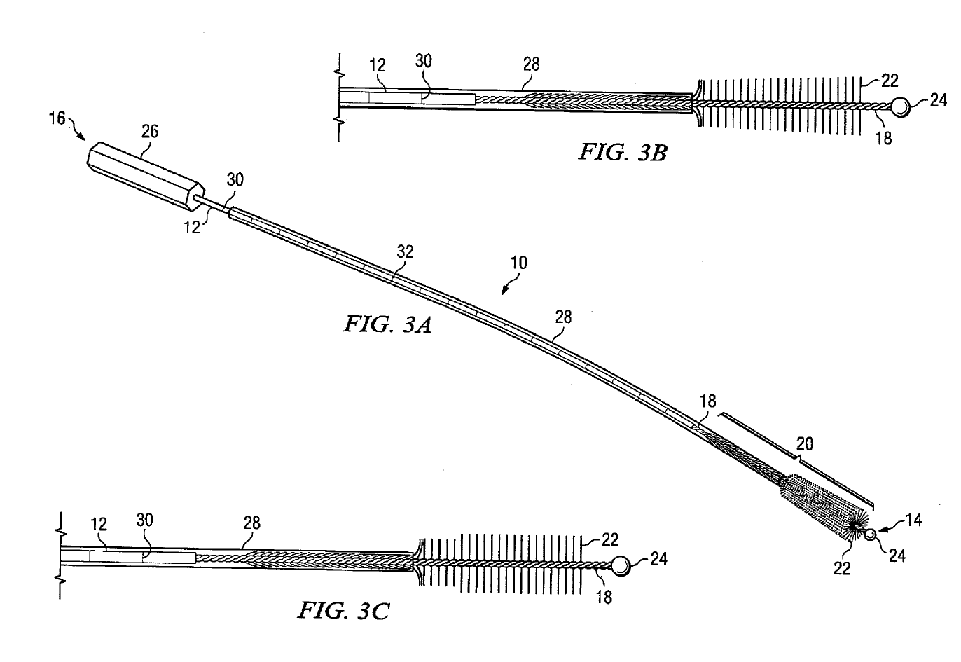

As shown more clearly FIGS. 1B and 1C, the diameter of the bristles 22 about the distal end 14 of the wire 18 may be tapered smoothly or in a step fashion, respectively. It has been found that the conically tapered brush bristles 22 may be easier to manufacture in some systems, but a step tapered brush bristles 22 pattern may be desired by some practitioners to give more certainty on the length of bristles 22 exposed during partial exposure of the bristles 22 as described with reference to FIG. 3.

In the tapered pattern of FIG. 1 B, the bristles 22 extend approximately 2 mm to 4 mm from the wire 18 to give a brush diameter of approximately 4 mm to 8 mm. The bristles extend approximately 5 cm to 7 cm along the wire 18 to give the practitioner the option of exposing a long or short brush, as discussed below. While one skilled in the art will understand that varying the brush length in small increments will not cause a discernible loss of functionality, a brush of this length has been found to have advantages over shorter brushes, especially when included with tapered bristles, such as more accurate tissue collection.

The stepped embodiment of FIG. 1 C includes bristles 22 that begin approximately 5 mm from the atraumatic bulb 24 on distal end 14 of wire 18. The bristles 22 have a length that forms a brush 20 first diameter of approximately 8 mm and cover approximately 2 cm of the distal end 14 of the wire 18, then step down to bristles 22 having a length that forms a brush 20 second diameter of approximately 7 mm that cover the next approximately 2 cm of the distal end 14 of the wire 18, and finally step down to bristles 22 having a length that forms a brush 20 third diameter of approximately 6 mm that cover the next approximately 2 cm of the distal end 14 of the wire 18. These dimensions are provided as an embodiment only and may be adjusted to include more or less steps to provide the desired brush shape.

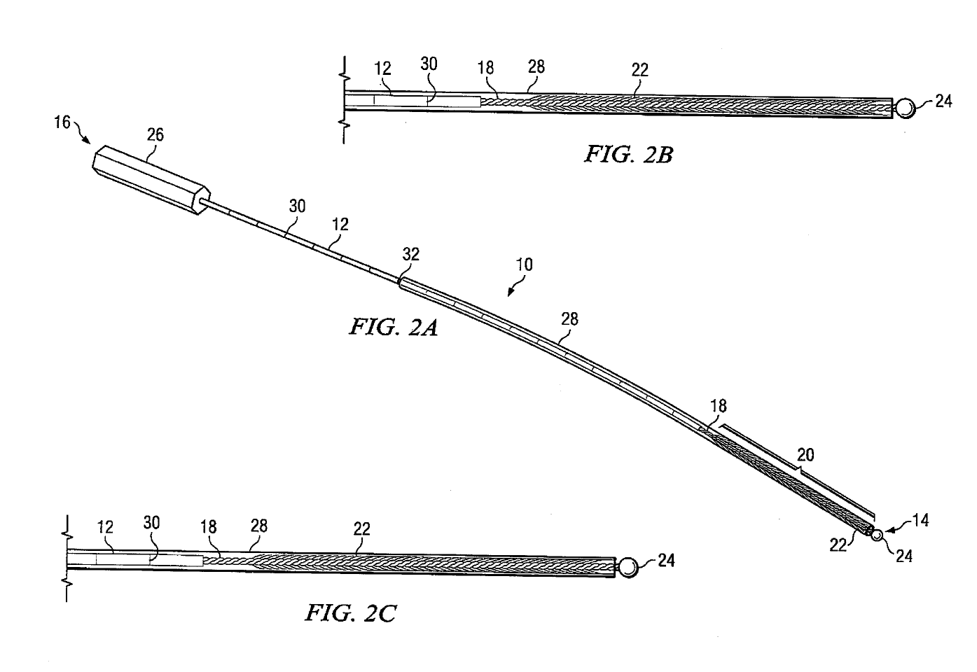

Turning now to FIG. 2, FIG. 2A is a perspective view of a tissue sampling device with a sheath 28 positioned to cover the brush bristles, FIG. 2B is a close up of the distal of the device in FIG. 2A with conically tapered brush bristles, FIG. 2C is a close up of the distal of the device in FIG. 2A with step tapered brush bristles. In accordance with this embodiment, the tissue sampling device 10 includes a movable sheath 28 of shorter length than the wire 18 that slides over the tube 12 to allow for selective covering of the bristles 22. The sheath 28 in the present embodiment is approximately 21 cm in length. While the sheath 28 may be of differing lengths without losing its functionality, the sheath 28 should be shorter than the wire 18 by the length of the brush 20. The sheath 28 can be moved laterally either towards the distal end 14 or proximal end 16 of the wire 18. When moved toward the distal end 14 of the wire 18, the sheath 28 covers the brush 20. Therefore, before inserting the tissue sampling device 10 for collection, the sheath 28 can be moved to the distal end 14 of the wire 18 to protect the brush 20 from errant tissue contamination during insertion.

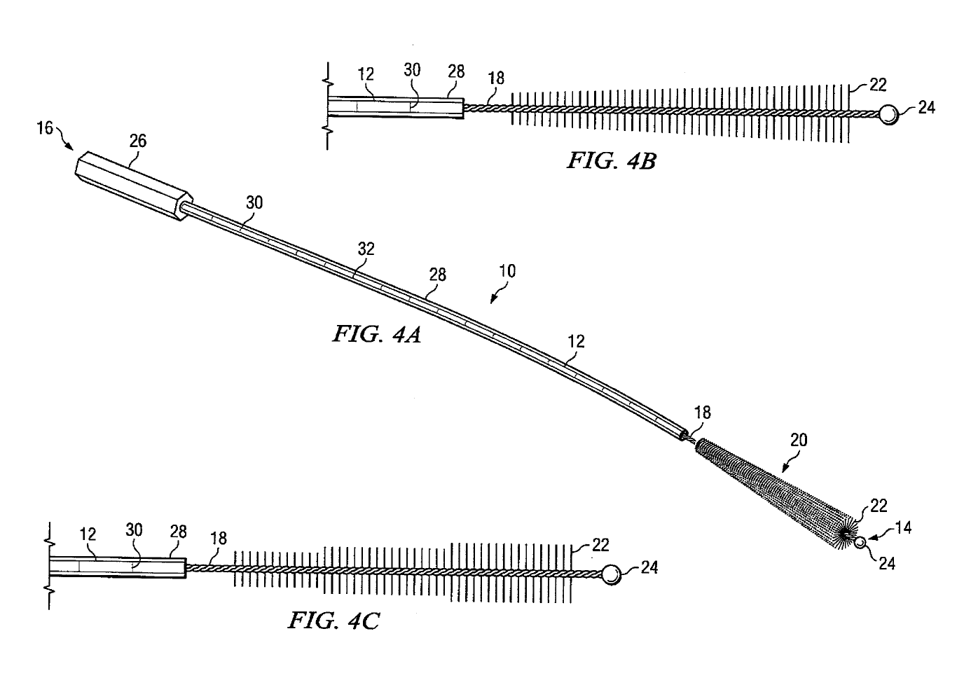

When the sheath 28 covers the brush 20 during insertion, the bristles 22 of the brush 20 are pressed against the steel wire 18 in the distal end 14 direction, providing a small diameter insertion device which improves comfort for the patient. While covered during insertion, the bristles 22 are also protected from collecting tissue from unwanted areas, reducing the risk of misleading results. After the device 10 has been inserted to a proper depth for tissue collection, the sheath 28 can be moved in the proximal end 16 direction to expose the bristles 22 of the brush 20 for proper tissue collection, as shown in FIGS. 3 and 4. Once tissue collection is complete, the sheath 28 can be moved back to the distal end 14 of the tube 12 so that the bristles 22 and associated tissue sample are protected upon removal. The sheath 28 should have an inside diameter just larger than the outside diameter of the tube 12 so that movement of the sheath 28 along tube 12 can be made with ease. Similar to the tube 12, the sheath 28 should be made of a material that is rigid enough to keep its shape during insertion of the device 10 and during the opening and closing of the sheath 28 during tissue collection. However, it should be flexible so as not to cause discomfort for the patient. In contrast to tube 12, it is desirable for sheath 28 to be clear, allowing the practitioner to see any markings on tube 12 while tube 12 is covered by sheath 28. One skilled in the art will understand that the sheath 28 can be made of plastic, other polymers approved by the FDA for such uses, and the like.

Another reason to have a transparent sheath 28 is to adjust the exposure of brush 20 during use as described below. In addition to gradations 30, a sheath mark 32 may be added along tube 12 to indicate the proximal end of sheath 28 when brush 20 is fully covered. As sheath 28 is moved in a proximal end 16 direction, the amount of brush uncovered may be determined by reference to the distance between the proximal end 16 of the sheath 28 and the sheath mark 32. Combining the sheath mark 32, transparent sheath 28, and a longer brush 20 with a wide range of bristles 22 lengths allows the practitioner to use a single brush on a wide variety of anatomies without compromising either patient comfort or collection effectiveness. In particular, the uterus 34 has a generally conical shape, although the size will vary from patient to patient. The tapered brush 20 is longer in the area of the uterus 34 where a longer brush is easily tolerated by the patient and shorter where a longer brush would be uncomfortable and sample unwanted areas.

Referring now to FIGS. 3 and 4, FIG. 3 shows an embodiment with a portion of the brush 20 exposed, while FIG. 4 shows the entire brush 20 exposed. In particular, FIG. 3A is a perspective view of a tissue sampling device 10 with the sheath 28 partially pulled back from the brush bristles 22, FIG. 3B is a close up of the distal end 14 of the device 10 in FIG. 3A with conically tapered brush bristles 22, FIG. 3C is a close up of the distal end 14 of the device 10 in

FIG. 3A with step tapered brush bristles 22. In contrast, FIG. 4A is a perspective view of a tissue sampling device 10 with the sheath 28 pulled all the way back to fully expose the brush bristles 22, FIG. 4B is a close up of the distal end 14 of the device 10 in FIG. 4A with conically tapered brush bristles 22, FIG. 4C is a close up of the distal end 14 of the device 10 in FIG. 4A with step tapered brush bristles 22. The contrast between FIG. 3 and FIG. 4 shows that a range of brush 20 may be exposed to accommodate differing patient anatomy or differing sample goals as discussed further below.

In accordance with one method, a tissue sampling device 10 is provided having an elongated plastic tube 12 covering a portion of a spirally twisted wire 18 having opposed proximal 16 and distal 14 ends, wherein the plastic tube 12 is marked in gradations 30 along the length of the plastic tube 12. The distal end 14 of the plastic wire 18 contains a brush 20 approximately 5 cm to 7 cm along the distal end 14 of the tube 12 and ending approximately one-half cm from the distal end 14 of the tube 12. The brush 20 includes bristles 22 fixed within the spirally wrapped wire 18 about the diameter of the distal end 14 of the wire 18 protruding beyond the plastic tube 12 for collection of a tissue sample. The diameter of the bristles 22 about the distal end 14 of the wire 18 is approximately 6 to 8 mm and the bristles 22 are tapered from the distal end 14 of the wire 18, in either a smooth or stepped fashion. The device 10 also including an atraumatic bulb 24 on the distal end 14 of the wire 18 and the proximal end 16 of the wire 18 includes a handle 26. A movable sheath 28 of shorter length than the wire 18 is enclosed around the tube 12.

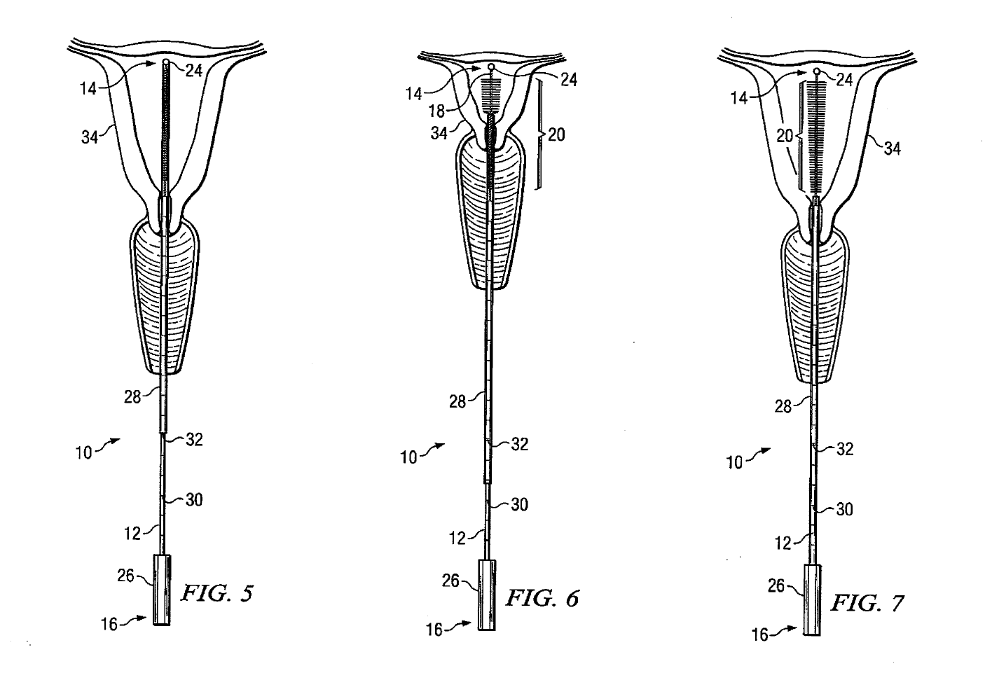

Before insertion, the sheath 28 is moved along the tube 12 to the distal end 14 of the wire 18 to about the atraumatic bulb 24 thereby covering the bristles 22 of brush 20. Because the bristles 22 of the brush 20 extend in a radial direction, the bristles 22 lay against the wire 18 during insertion and removal to protect the bristles 22 from damage, and also protect the sample collected on the bristles 22. This improves comfort for the patient and protects the brush 20 upon insertion and removal. The distal end 14 of the device 10 is then inserted into an area of tissue where a sample is to be taken, such as uterus 34 as shown in FIG. 5. Using the gradations 30 along the tube 12, the insertion depth is measured to assure accurate placement of brush 20 within uterus 34. The sheath 28 is then moved in the proximal end 16 direction to expose at least a portion of the brush 20 as shown in FIG. 6. In particular, FIG. 6 depicts a uterus 34 of relatively smaller size and shows how partial exposure of a tapered brush 20 protects the comfort of the patient and accurate sampling. The length of brush 20 exposed is determined by the distance sheath 28 is pulled in a proximal end 16 direction. In a larger uterus 34, more of the brush 20 may be exposed, or even the entire brush as shown in FIG. 7. The amount of brush being exposed may be determined by reference to the distance between the proximal end of the sheath 28 and the sheath mark 32. The device 10 is then rotated using the handle 26 to collect a tissue sample. Before removing the device 10, the sheath 28 is once again moved in the distal end 14 direction to the bulb 24 located on the distal end 14 of the wire 12, such that the proximal end 16 of the sheath 28 aligns with the sheath mark 32. This enables protection of the sample upon removal of the device 10. The device 10 is then removed from the subject, and the tissue sample is collected from the brush 20.

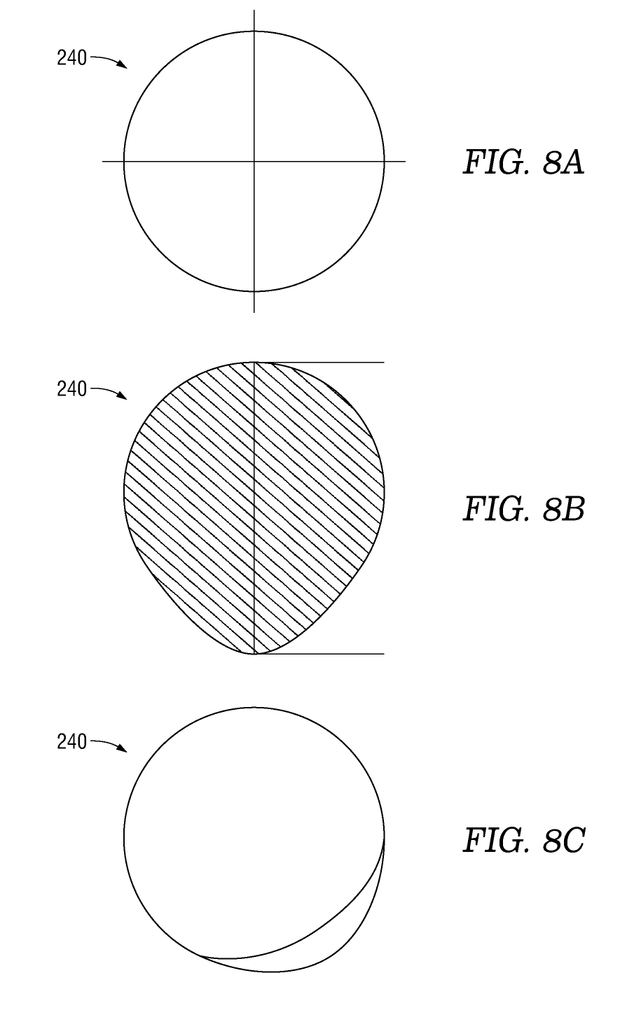

Thus far, the atraumatic bulb 24 has been depicted as having a round or spherical shape for ease of entry of the device 10 into the cervical area. In some embodiments, to further facilitate insertion of the device 10 into the cervical area, the shape of the atraumatic bulb may be designed so that it is more elongated than round when looking at the profile or side view of the bulb. FIGS. 8A-8C illustrate a top view, cross-sectional view, and perspective view, respectively, of an elongated atraumatic bulb 240 in accordance with these embodiments. As can be seen, the atraumatic bulb 240 has an egg-shaped profile wherein one end of the atraumatic bulb 240 is narrower (i.e., has a smaller radius) than the other end. The narrower end of the atraumatic bulb 240 may then be used as the insertion end for easing the device 10 more gently into the cervical area. It is of course possible to reverse the atraumatic bulb 240 so that the broader end is used as the insertion end without departing from the scope of the disclosed embodiments. Likewise, it is also possible to use other elongated shapes for the atraumatic bulb besides the egg-shaped profile shown in FIGS. 8A-8C without departing from the disclosed embodiments. Examples of other elongated shapes may include elliptical, oblong, ovate, ovoid, pear-shaped, bullet-shaped, and the like.

Even though many of the examples discussed herein are applications of the present devices and methods as used for collecting endometrial cells, the present devices and methods also can be applied for collecting tissue samples in other areas of the body or from non-human patients.

While the invention has been described with reference to one or more particular embodiments, those skilled in the art will recognize that many changes may be made thereto without departing from the spirit and scope of the description. Each of these embodiments and obvious variations thereof is contemplated as falling within the spirit and scope of the claimed invention, which is set forth in the following claims.

What is claimed is:

1. A tissue sampling device comprising:
a flexible spirally wrapped wire having opposed proximal and distal ends;
a flexible tube covering a portion of the wrapped wire to provide increased rigidity, the tube being marked with gradations;
a brush located at the distal end of the wire, the brush comprising bristles fixed within the spirally wrapped wire;
a sheath slidable along the flexible tube to selectively cover and uncover the brush;
an atraumatic bulb is fixed to the distal end of the wire; and
a handle is fixed to the proximal end of the wire;
wherein the atraumatic bulb has a profile having one of the following shapes: round, egg-shaped, elliptical, oblong, ovate, ovoid, pear-shaped, or bullet-shaped.

2. The tissue sampling device according to claim 1 wherein:
the brush is tapered such that the bristles are longest near the distal end of the twisted wire; and
the brush is greater than 5 cm in length.

3. The tissue sampling device according to claim 1 wherein:
the diameter of the brush is tapered from approximately 4 mm to approximately 8 mm, with the larger diameter near the distal end of the wire.

4. The tissue sampling device according to claim 1 wherein:
the diameter of the brush is tapered from approximately 4 mm to approximately 8 mm, with the larger diameter near the distal end of the wire;
the brush being at least 5 cm long; and
the wire is approximately 28.5 centimeters in length.

5. The tissue sampling device according to claim 1 wherein:
the sheath is of a shorter length than the wrapped wire and slides over the flexible tube wherein the sheath can be moved along the flexible tube to cover the brush during insertion, uncover the brush for collection, and cover the brush during removal.

6. The tissue sampling device according to claim 1 wherein:
the sheath is transparent.

7. The tissue sampling device according to claim 1 wherein:
the gradations are labeled to indicate the distance from the distal end of the spirally wrapped wire.

8. The tissue sampling device of claim 5 further including:
a mark on the tube indicating a proximal end of the sheath when the sheath is slid over the brush.

9. The tissue sampling device according to claim 1 wherein:
the flexible tube is scored in centimeter gradations and labeled at least every other centimeter from the distal end of the flexible tube to the proximal end of the flexible tube.

10. The tissue sampling device of claim 9 further including:
a marking on the flexible tube indicating a proximal end of the sheath when the sheath is slid over the brush.

11. The tissue sampling device according to claim 1 wherein:
the brush is spaced about one-half centimeter from the atraumatic bulb, the brush having a first diameter of about 8 millimeters closest to the atraumatic bulb, followed by a second diameter of about 7 millimeters, followed by a third diameter of about 6 millimeters, forming a stepped brush.

12. A tissue sampling device according to claim 7 wherein:
the brush is spaced about one-half centimeter from the atraumatic bulb, the brush having a first diameter of about 8 millimeters closest to the atraumatic bulb for a length of about 2 centimeters, followed by a second diameter of about 7 millimeters for a length of about 2 centimeters, followed by a third diameter of about 6 millimeters for a length of about 2 centimeters, forming a stepped brush. Patent

13. A tissue sampling device comprising:
a spirally wrapped wire having opposed proximal and distal ends;
a flexible tube covering a portion of the spirally wrapped wire;
the flexible tube;
a brush located near the distal end of the wire, the brush being about 5.5 cm long and ending approximately one-half cm from the distal end of the wire, the brush comprising bristles, wherein the diameter of the brush about the distal end of the wire is approximately 4 mm to 8 mm and tapered from the distal end of the wire;
an atraumatic bulb fixed on the distal end of the wire, the atraumatic bulb having a profile that has one of the following shapes: round, egg-shaped, elliptical, oblong, ovate, ovoid, pear-shaped, and bullet-shaped; and
a sheath movable along the flexible tube from a position covering the brush to a position exposing at least a portion of the brush.

14. The tissue sampling device of claim 13 further including a mark on the tube indicating a proximal end of the sheath when the sheath is moved over the brush.

15. The tissue sampling device according to claim 13 wherein the brush starts from about one-half centimeter from the atraumatic bulb, the brush having a first diameter of about 8 millimeters closest to the atraumatic bulb, followed by a second diameter of about 7 millimeters, followed by a third diameter of about 6 millimeters, forming a stepped brush.

16. The tissue sampling device according to claim 13 wherein the flexible tube has gradations.

17. A method of collecting a tissue sample comprising the steps of:
providing a tissue sampling device having an elongated flexible tube covering a portion of a spirally wrapped wire having opposed proximal and distal ends, wherein the flexible tube is scored in gradations and labeled from the distal end to the proximal end of the flexible tube, wherein the distal end of the wire contains a brush at least 5.5 centimeters in length, the brush comprising bristles, wherein the diameter of the brush is about 4 mm to 8 mm and tapered from the distal end of the wire, the device also including an atraumatic bulb on the distal end of the wire, and a sheath slidable over the tube from a position abutting the atraumatic bulb and covering the brush to positions where portions of the brush are exposed, including a position where the entirety of the brush is exposed;
inserting the distal end of the tissue sampling device into an area of tissue where a sample is to be taken, the distal end having a profile that has one of the following shapes:
round, egg-shaped, elliptical, oblong, ovate, ovoid, pear-shaped, and bullet-shaped;
measuring the depth of insertion of the tissue sampling device after the inserting step by observing the gradations along the flexible tube;
moving the sheath proximally along the plastic tube to expose at least a portion of the brush;
operating the tissue sampling device to collect a tissue sample;
moving the sheath distally along the flexible tube to cover the brush and protect the tissue sample; and
removing the tissue sampling device from the subject.

18. The method of collecting a tissue sample according to claim 17 further comprising the step of collecting a tissue sample from the tissue sampling device.

19. The method of collecting a tissue sample according to claim 17 further comprising the step of:
measuring the length of brush exposed by noting the passage of the proximal end of the sheath over the gradations on the flexible tube.

20. The method of collecting a tissue sample according to claim 17 wherein the tissue sampling device in the providing step also includes a mark along the flexible tube indicating the proximal end of the sheath when the sheath is fully covering the brush and a sheath that is at least translucent such that the mark is visible beneath the sheath, wherein the method further comprises the step of:
measuring the length of brush exposed by noting the distance between the proximal end of the sheath and the mark.
